(19) United States
(12) Patent Application Publication
(10) Pub. No.: US 2013/0084257 A1
(43) Pub. Date: Apr. 4, 2013

(54) TRANSDERMAL ABSORPTION PROMOTER, AND EXTERNAL SKIN FORMULATION THEREOF

(71) Applicants: Kenya Ishida, Hiratsuka-shi (JP); Yasuko Obata, Tokyo (JP); Kozo Takayama, Tokyo (JP)

(72) Inventors: Kenya Ishida, Hiratsuka-shi (JP); Yasuko Obata, Tokyo (JP); Kozo Takayama, Tokyo (JP)

(73) Assignee: TAKASAGO INTERNATIONAL CORPORATION, Tokyo (JP)

(21) Appl. No.: 13/703,827

(22) PCT Filed: Jun. 16, 2011
(86) PCT No.: PCT/JP2011/064322

§ 371 Date: Dec. 12, 2012

(30) Foreign Application Priority Data
Jun. 16, 2010 (JP) 2010-137349

(51) Int. Cl. A61K 31/075; A61K 31/08; A61K 31/045

(52) U.S. Cl. CPC A61K 31/075; A61K 31/045; A61K 31/08
USPC 424/62; 568/828; 514/729; 568/671; 514/723; 568/670; 514/715

(57) ABSTRACT

The present invention provides a substance which promotes the transdermal absorption of a pharmacologically active component while little irritating the skin. The present invention relates to a transdermal absorption promoter which comprises, as the active component, at least one member selected from among isopulegol, 2-(menthoxy)ethanol and 2-methyl-3-(menthoxy)propane-1,2-diol; and an external skin formulation which comprises a pharmacologically active component such as a psychotropic component, an anti-inflammatory component, an analgesic component, an antipyretic component, a whitening component or a hair growth-promoting component, together with the aforesaid transdermal absorption promoter.

### TECHNICAL FIELD

The present invention relates to a transdermal absorption promoter for transdermally administering a pharmacologically active component and an external skin formulation comprising the transdermal absorption promoter. More specifically, it relates to a transdermal absorption promoter and an external skin formulation, which are excellent in both transdermal absorbability and safety, enables the quick delivery of a desired pharmacologically active component to a target site or throughout the whole body via the circulatory system, and, therefore, are efficacious for treating various diseases.

### BACKGROUND ART

In recent years, as the method for administrating a pharmaceutical, there has been developed transdermal therapeutic system (TTS) by which a desired drug is delivered via the skin to the whole body so that the drug can exert its therapeutic effect over a long period of time. For example, nitroglycerin and isosorbide nitrate for treating angina, chlonidine for treating hypertension, estradiol for treating menopausal disorders and so on have been already applied to TTS in practice. Compared with oral administration or injection method, however, transdermal administration of a drug is disadvantageous in that a pharmacologically active component of the drug is absorbed at an extremely low level. This problem becomes particularly serious when the pharmacologically active component is soluble in water. Therefore, attempts have been eagerly made to develop transdermal absorption promoters capable of acting on the horny layer, which serves as a barrier against the transdermal absorption of a drug, and thus lowering the barrier function of the skin to thereby enhance the transdermal absorbability of the drug.

Known examples of these transdermal absorption promoters include aprotic solvents such as dimethyl sulfoxide and N,N-dimethylformamide (Patent Literature 1), anionic or amphoteric surfactants (Patent Literatures 2 and 3) and 1-dodecylazacycloheptan-2-one (AZONE) (Patent Literature 4). Further, there are known terpene compounds including terpene ketones such as 1-carbone, menthone and piperitone (Patent Literature 5) and d-limonene (Patent Literature 6). Furthermore, there are known p-menthane derivatives such as 1-menthol (Patent Literature 7), p-menthane-3,8-diol and 3-1-menthoxy-1,2-diol (Patent Literatures 8 and 9) and N-substituted-p-menthane-3-carboxamide (Patent Literature 10).

In addition, glycols, fatty acids such as oleic acid, fatty acid esters such as isopropyl myristate and isopropyl palmitate, etc. have been reported.

### CITATION LIST

### Patent Literature

PTL 1 U.S. Pat. No. 3,551,554
PTL 2 JP-A-51-32724
PTL 3 JP-A-52-83914
PTL 4 JP-A-52-1035
PTL 5 JP-A-2-193932
PTL 6 JP-A-2-207024
PTL 7 JP-A-4-217926
PTL 8 JP-A-2000-143475
PTL 9 JP-A-2000-143543
PTL 10 JP-A-2001-58961

### SUMMARY OF INVENTION

### Technical Problem

However, the transdermal absorption promoters described in Patent Literature 1 to 10 are still insufficient from three viewpoints, namely, transdermal absorption-promoting effect, safety (for example, not irritating the skin) and usability (for example, having an offensive smell). Thus, it has been required to develop a transdermal absorption promoter which is safe, has good usability and exerts an excellent effect.

An object of the invention, which has been made by focusing attention on the problems occurring in external skin formulations as discussed above, is to provide a transdermal absorption promoter having an excellent transdermal absorption-promoting effect, a high safety and good usability, and an external skin formulation comprising the same.

### Solution to Problem

As the results of intensive studies to develop a transdermal absorption promoter having the desired characteristics as described above, the present inventors have found that isopulegol, 2-(menthoxy)ethanol and 2-methyl-3-(menthoxy)propane-1,2-diol remarkably promote the transdermal absorption of drugs. These are p-menthan derivatives and known as substances having cooling effect or substances having refreshing effect. For example, isopulegol is known as a substance having cooling effect (JP-A-6-65023), 2-(menthoxy)ethanol is known as a substance having a cooling effect and cooling persistent effect (JP-A-2005-343915) and 2-methyl-3-(menthoxy)propane-1,2-diol is known as a substance imparting a comfortable cool feeling or refreshing feeling (JP-A-7-82200). However, it has never been clarified that these substances have such remarkable transdermal absorption-promoting effect. Moreover, it has never been attempted to use these substances as a transdermal absorption promoter for the transdermal absorption of a pharmacologically active component. Namely, such an attempt has been made for the first time by the present inventors, and the present inventors have found that these have superior effects, compared to the conventionally known p-menthane derivatives.

The present invention encompasses the following.

[1] A transdermal absorption promoter, comprising, as an active component, at least one member selected from the group consisting of isopulegol, 2-(menthoxy)ethanol and 2-methyl-3-(menthoxy)propane-1,2-diol.

[2] The transdermal absorption promoter according to [1], which further comprises at least one component selected from the group consisting of menthol, menthone, camphor, pulegol, cineole, 3-menthoxypropane-1,2-diol, N-alkyl-p-menthane-3-carboxamide, 3-menthoxy-2-methylpropane-1,2-diol, p-menthane-3,8-diol, 3-menthoxypropan-1-ol, 4-1-menthoxybutan-1-ol (menthyl 3-hydroxybutanoate), menthyl 3-hydroxybutanoate, 1-(2-hydroxy-4-methyl-cyclohexyl)ethanone, menthyl lactate, menthol glycerol ketal, N-methyl-2,2-isopropylmethyl-3-methylbutaneamide, menthyl glyoxylate, menthyl succinate, menthyl glutarate, peppermint oil, spearmint oil, eucalyptus oil and mint oil.

[3] The transdermal absorption promoter according to [1] or [2], which further comprises at least one warming substance selected from the group consisting of vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, 4-(1-menthoxy-methyl)-2-(3′-methoxy-4′-hydroxyphenyl)-1,3-dioxolane, red pepper oil, red pepper oleoresin, nonylic acid vanillylamide, jambu oleoresin, Japanese pepper extract, sanshool-I, sanshool-II, sanshoamide, black pepper extract, chavicine, piperine and spilantol.

[4] An external skin formulation, which comprises 0.01 to 50 mass % of the transdermal absorption promoter according to any one of [1] to [3].

[5] The external skin formulation according to [4], which comprises at least one pharmacologically active component selected from the group consisting of a psychotropic component, an anti-inflammatory component, an analgesic component, an antipyretic component, a whitening component and a hair growth-promoting component.

[6] A method for enhancing/controlling the transdermal permeability of at least one pharmacologically active component, which is selected from the group consisting of a psychotropic component, an anti-inflammatory component, an analgesic component, an antipyretic component, a whitening component and a hair growth-promoting component, the method comprising using, as an active component, the transdermal absorption promoter according to any one of [1] to [3].

[7] A method for controlling a cooling effect, which comprises using, as an active component, the transdermal absorption promoter according to any one of [1] to [3].

[8] At least one member selected from the group consisting of isopulegol, 2-(menthoxy)ethanol and 2-methyl-3-(menthoxy)propane-1,2-diol for use in transdermal absorption promotion.

### Advantageous Effects of Invention

By using the transdermal absorption promoter and external skin formulation according to the invention, the transdermal absorption of a drug is remarkably enhanced by isopulegol, 2-(menthoxy)ethanol or 2-methyl-3-(menthoxy)propane-1,2-diol contained therein. Further, the transdermal absorption promoter and external skin formulation according to the invention have little smell and high safety, e.g., not irritating the skin, and it becomes possible to impart the desired refreshing feeling and warm feeling. That is, the external skin formulation comprising the transdermal absorption promoter according to the invention is highly useful in treating various diseases, since it is excellent in safety and usability and enables the quick delivery of a desired drug or pharmacologically active component to a target site or throughout the whole body via the circulatory system.

### DESCRIPTION OF EMBODIMENTS

Isopulegol that is to be used in the transdermal absorption promoter and external skin formulation according to the invention may be a racemic form thereof or an optical isomer thereof. As a preferred optical isomer thereof, 1-(−)-isopulegol can be exemplified.

2-(Menthoxy)ethanol that is to be used in the invention may be a racemic form thereof or an optical isomer thereof. As a preferred optical isomer thereof, 2-(1-menthoxy)ethanol can be exemplified.

2-Methyl-3-(menthoxy)propane-1,2-diol that is to be used in the invention may be a racemic form thereof or an optical isomer thereof. As a preferred optical isomer thereof, 2-methyl-3-(1-menthoxy)propane-1,2-diol can be exemplified.

Either one of isopulegol, 2-(menthoxy)ethanol and 2-methyl-3-(menthoxy)propane-1,2-diol or a combination of two or more thereof may be used.

In addition to isopulegol, 2-(menthoxy)ethanol and 2-methyl-3-(menthoxy)propane-1,2-diol, it is possible in the invention to use other substances having cooling effect or substances having refreshing effect to thereby prepare a transdermal absorption promoter having an enhanced transdermal absorption-promoting effect and imparted desired refreshing feeling and warm feeling.

As these substances having cooling effect or substances having refreshing effect, which are to be used in addition to isopulegol, 2-(menthoxy)ethanol and 2-methyl-3-(menthoxy)propane-1,2-diol, any substances including publicly known or well-known cooling substances or refreshing substances may be used without specific restriction. Examples thereof include menthol, menthone, camphor, pulegol, cineole, mint oil, 3-menthoxypropane-1,2-diol, N-alkyl-p-menthane-3-carboxamide, 3-menthoxy-2-methylpropane-1,2-diol, p-menthane-3,8-diol, 3-menthoxypropan-1-ol, 4-1-menthoxybutan-1-ol (menthyl 3-hydroxybutanoate), menthyl 3-hydroxybutanoate, 1-(2-hydroxy-4-methyl-cyclohexyl)ethanone, menthyl lactate, menthol glycerol ketal, N-methyl-2,2-isopropylmethyl-3-methylbutaneamide, menthyl glyoxylate, menthyl succinate, menthyl glutarate, peppermint oil, spearmint oil, eucalyptus oil and mint oil. Either one of these substances or a combination of two or more thereof may be used.

In the invention, isopulegol, 2-(menthoxy)ethanol and 2-methyl-3-(menthoxy)propane-1,2-diol and other substances having cooling effect or substances having refreshing effect may be used at an arbitrary ratio so long as the advantages of the invention are not impaired. It is usually preferred that the substances having cooling effect or substances having refreshing effect, which are to be used in addition to isopulegol, 2-(menthoxy)ethanol and 2-methyl-3-(menthoxy)propane-1,2-diol, are blended in an amount of 0.001 to 10 times, preferably 0.01 to 5 times, as much as the amount of isopulegol, 2-(menthoxy)ethanol and 2-methyl-3-(menthoxy)propane-1,2-diol.

In the invention, it is also possible to use, as an additional component, a warming substance, thereby preparing a transdermal absorption promoter having an enhanced transdermal absorption-promoting effect and imparted desired warm feeling or refreshing feeling.

As the warming substance, any substances having a warming effect including publicly known or well-known warming substances may be used without specific restriction. Examples thereof include vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, 4-(1-menthoxy-methyl)-2-(3′-methoxy-4′-hydroxyphenyl)-1,3-dioxolane, red pepper oil, red pepper oleoresin, nonylic acid vanillylamide, jambu oleoresin, Japanese pepper extract, sanshool-I, sanshool-II, sanshoamide, black pepper extract, chavicine, piperine and spilantol. Either one of these substances or a combination of two or more thereof may be used.

In the invention, the warming substance may be used at an arbitrary ratio so long as the advantages of the invention are not impaired. Usually, the warming substance is employed in an amount of 0.0001 to 10 times, preferably 0.001 to 5 times, as much as the amount of isopulegol, 2-(menthoxy)ethanol and 2-methyl-3-(menthoxy)propane-1,2-diol.

The pharmacologically active component to be used in the external skin formulation according to the invention is not specifically restricted, so long as the combined use thereof with isopulegol, 2-(menthoxy)ethanol or 2-methyl-3-(menthoxy)propane-1,2-diol can achieve a transdermal absorption-promoting effect. Namely, an appropriate pharmacologically active component may be selected from publicly known drugs. Examples of such pharmacologically active components include: medicative components, e.g., steroidal anti-inflammatory drugs such as prednisolone, dexamethasone, hydrocortisone, fluocinolone acetonide, betamethasone valerate, betamethasone dipropionate, clobetasone butyrate and prednisolone succinate, nonsteroidal anti-inflammatory agents and ester derivatives thereof such as indomethacin, diclofenac, ibuprofen, ketoprofen, flufenamic acid, ketorolac, flurbiprofen, felbinac, suprofen, pranoprofen, tiaprofen and loxoprofen, antiallergic drugs such as tranilast, azelastine, ketotifen, ibudilast, oxatomide and emedastin, antihistamine drugs such as diphenhydramine, chlorpheniramine, promethazine and tripelennamine, the central nervous system drugs such as chlorpromazine, nitrazepam, diazepam, phenobarbital and reserpine, psychotropic drugs such as fluvoxamine, paroxetine sertraline, milnacipran, venlafaxine, duloxetine, nefazodone, amitriptyline hydrochloride and imipramine hydrochloride, hormonal drugs such as insulin, testosterone, norethisterone, methyltestosterone, progesterone and estradiol, antihypertensive drugs such as clonidine, reserpine and guanethidine sulfate, cardiac stimulants such as digitoxin and digoxin, antiarrhythmic drugs such as propranolol hydrochloride, procainamide hydrochloride, ajmaline, pindolol and tulobuterol hydrochloride, coronary vasodilators such as nitroglycerin, isosorbide nitrate, papaverine hydrochloride and nifedipine, local anesthetics such as lidocaine, benzocaine, procaine hydrochloride and tetracaine, analgesic drugs such as morphine, aspirin, codeine, acetanilide, aminopyrine and antipyrine, antipyretic drugs such as indomethacin, salicylic acid, glycol salicylate, acetaminophen, diclofenac sodium, ibuprofen, sulindac, naproxen, ketoprofen, flufenamic acid, ibufenac, fenbufen, alclofenac, phenylbutazone, mefenamic acid, bendazac, piroxicam, flurbiprofen, pentazocine, buprenorphine hydrochloride and butorphanol tartrate, skeletal muscle relaxants such as eperisone, tizanidine, tolperisone, inaperisone and pridinol mesylate, antifungal drugs such as acetophenylamine, nitrofurazone, pentamycin, naphthiomate, miconazole, omoconazole, clotrimazole, butenafine hydrochloride and bifonazole, antineoplastic drugs such as 5-fluorouracil, busulfan, actinomycin, bleomycin and mitomycin, urinary incontinence drugs such as terolidine hydrochloride and oxybutynin hydrochloride, antiepileptic drugs such as nitrazepam and meprobamate, anti-Parkinsonism drugs such as chlorzoxazone and levodopa, Alzheimer-type dementia drugs such as rivastigmine, antiemetic drugs such as ondansetron and granisetron, drugs to assist in stopping smoking such as nicotine and, furthermore, vitamins, prostaglandins and so on; whitening components such as arbutin, labdenoic acid, kojic acid, ellagic acid, ascorbic acid, ascorbine derivatives, lactic acid, glycolic acid and tartaric acid; and hair growth-promoting components such as minoxidil, finasteride, isopropyl methylphenol, gingko extract, carpronium chloride, diphenhydramine hydrochloride, Polygonum root, glycyrrhizic acid (dipotassium glycyrrhizinate), dialkylmonoamine derivatives, powdered ginger, ginger, cepharanthine, cnidium rhizome, swertia, panax rhizome, ginseng, capsicum tincture, hinokitiol, placenta extract and pentadecanoic acid glyceride. Needless to say, the invention is not restricted to these components.

The content of isopulegol, 2-(menthoxy)ethanol or 2-methyl-3-(menthoxy)propane-1,2-diol in the external skin formulation according to the invention is in the range of 0.01 to 50 mass %, preferably 0.1 to 20 mass % and still more preferably 0.5 to 10 mass %, relative to the total amount of the formulation. When the content thereof is less than 0.01 mass %, the transdermal absorption-promoting effect can not be fully exerted. When the content exceeds 50 mass %, the effect can not be improved anymore and the formulation becomes unstable in some cases. The external skin formulation according to the invention may be formulated into arbitrary dosage forms commonly employed for external formulations such as ointments, creams, gels, gel-type creams, lotions, sprays, cataplasms, tapes, reservoir type patches and so on.

The external skin formulation according to the invention can be produced by a common method by blending, as a transdermal absorption promoter, an appropriate amount of isopulegol, 2-(menthoxy)ethanol or 2-methyl-3-(menthoxy)propane-1,2-diol to a formulation. When isopulegol, 2-(menthoxy)ethanol or 2-methyl-3-(menthoxy)propane-1,2-diol is not easily soluble in a base, a solvent may be optionally used to improve the solubility. Next, the external skin formulation of the invention in the form of a cataplasm and a tape will be described in greater detail. In the cataplasm, for example, it is preferred to employ a hydrophilic base prepared by using a water-soluble polymer, a polyhydric alcohol and water while taking temporal stability, releasability, temporal absorbability and safety to the skin into consideration.

As the water-soluble polymer to be used in the hydrophilic base, one or more members may be appropriately selected from among gelatin, casein, pullulan, dextran, sodium alginate, soluble starch, carboxy starch, dextrin, carboxymethylcellulose, carboxymethylcellulose sodium, methylcellulose, ethylcellulose, hydroxyethylcellulose, polyvinyl alcohol, polyethylene oxide, polyacrylic acid, polyacrylamide, sodium polyacrylate, polyvinylpyrrolidone, carboxy vinyl polymer, polyvinyl ether, methoxyethylene-maleic anhydride copolymer, isobutylene-maleic anhydride copolymer, N-vinyl acetamide, N-vinyl acetamide-acrylic acid and/or acrylate copolymers and so on. In this case, the content of the water-soluble polymer is preferably in the range of 1 to 30 mass %, more preferably 1 to 20 mass % and still preferably 1 to 15 mass %, relative to the whole formulation. When the content thereof is less than 1 mass %, the viscosity may become too low and thus the formulation may not retain its shape. When the content thereof exceeds 30 mass %, the viscosity may become too high and thus the handling properties in kneading and coating may be deteriorated.

As the polyhydric alcohol, one or more members may be appropriately selected from among polyethylene glycol, propylene glycol, dipropylene glycol, polypropylene glycol, 1,3-butylene glycol, 1,4-butylene glycol, isobutylene glycol, glycerin, diglycerin, sorbitol and so on. The content of the polyhydric alcohol is preferably in the range of 5 to 90 mass %, more preferably 10 to 70 mass % and still preferably 20 to 60 mass %. When the content thereof is less than 5 mass %, insufficient moisturizing effect may be obtained. A content thereof exceeding 90 mass % may affect the solubility of the water-soluble polymer. The content of water is preferably in the range of 10 to 90 mass % and more preferably 20 to 80 mass %. Water is preferably used for dissolving the water-soluble polymer therein to induce thickness, aggregation and shape-retainability.

In addition to the essentially required components as discussed above, a crosslinking agent may be used, if necessary. Examples of the crosslinking agent include polyvalent metal compounds, such as aluminum hydroxide, aluminum chloride, calcium hydroxide, calcium chloride, aluminum sulfate, aluminum ammonium sulfate, aluminum potassium sulfate, magnesium metasilicate aluminate and dihydroxyaluminum aminoacetate; compounds having at least two epoxy groups in the molecule, such as ethylene glycol diglycidyl ether, polyethylene glycol diglycidyl ether, propylene glycol diglycidyl ether, polypropylene glycol diglycidyl ether, polytetramethylene glycol diglycidyl ether, glycerol polyglycidyl ether, polyglycerol polyglycidyl ether, sorbitol polyglycidyl ether, sorbitan polyglycidyl ether, trimethylolpropane polyglycidyl ether, pentaerythritol polyglycidyl ether, resorcin diglycidyl ether, neopentylglycol diglycidyl ether and 1,6-hexanediol diglycidyl ether. Either one of these crosslinking agent or a combination of two or more thereof may be appropriately used.

In addition, it is possible to add one or more components selected from among fillers such as kaolin, zinc oxide, titanium oxide, talc, bentonite and synthetic aluminum silicate, preservatives such as thymol, methylparaben and ethylparaben, antioxidants such as ascorbic acid, stearic acid esters, dibutyl hydroxytoluene, butyl hydroxyanisole, gallic acid esters, vitamin E, vitamin E acetate and disodium edetate, UV-absorbers such as 2-hydroxy-4-methoxybenzophenone, ethyl p-aminobenzoate, 2-(2-hydroxy-5-methylphenyl)benzotriazole, glycol salicylate, methyl salicylate and phenyl salicylate, and emulsifiers such as sorbitan fatty acid esters, glycerol fatty acid esters, decaglycerol fatty acid esters, polyoxyethylene sorbitan fatty acid esters, polyethylene glycol fatty acid esters and polyoxyethylene alkyl ethers.

As the support of this cataplasm, it is important to select a material not affecting the release of the pharmacologically active component. In other words, it is essentially required to use a support that neither interacts with the pharmacologically active component nor adsorb the same. For example, use can be made of films or sheets such as polyethylene, polypropylene, polyvinyl chloride, polyester, nylon and polyurethane, porous articles and expanded articles thereof, fabrics, non-woven fabrics and laminates of films or sheets together with porous articles, expanded articles, fabrics or non-woven fabrics. As a release coat, polyethylene, polypropylene, polyester and those treated with silicone, release paper and so on.

Next, a method for producing the cataplasm will be described in greater detail. The cataplasm, which comprises the transdermal absorption promoter according to the invention, can be easily produced in accordance with a publicly known production method. For example, a water-soluble polymer is mixed with a polyhydric alcohol and water and dispersed and dissolved therein to give a homogeneous kneaded product. If necessary, a stabilizer, an antioxidant, a UV absorber, an emulsifier, a preservative, an antiseptic, a perfume and so on are added thereto. Next, a pharmacologically active component and isopulegol, 2-(menthoxy)ethanol or 2-methyl-3-(menthoxy)propane-1,2-diol are added thereto and homogeneously dispersed. Then, the dispersion thus obtained is spread directly on a support. Alternatively, the dispersion may be once spread on a release-treated paper or film and then press-transferred onto the support. In the production method as described above, the order of adding the base, pharmacologically active component and other components is illustrated only by way of example. That is, the invention is not restricted to this order.

As the pressure-sensitive adhesive base of the tape, a material may be selected from publicly known ones by taking safety to the skin, release properties of the pharmacologically active component, stickiness to the skin and so on into consideration. As preferred pressure-sensitive adhesives, acryl-based pressure-sensitive adhesives, rubber-based pressure-sensitive adhesives, silicone-based pressure-sensitive adhesives and so on may be exemplified. As the acryl-based pressure-sensitive adhesives, homopolymers or copolymers of an alkyl (meth)acrylate having 4 to 18 carbon atoms or copolymers of the aforesaid alkyl (meth)acrylate with another functional monomer can be appropriately used.

Examples of the rubber-based pressure-sensitive adhesives include natural rubber, synthetic isoprene rubber, polyisobutylene, polyvinyl ether, polyurethane, polyisoprene, polybutadiene, styrene-butadiene copolymer, styrene-isoprene copolymer, styrene-isoprene-styrene block copolymer and so on.

As the silicone-based pressure-sensitive adhesives, those comprising polyorganosiloxane or polydimethylsiloxane as the main component may be used. Examples of a tackifier include rosin-based components such as hydrogenated, heterogenized, polymerized or esterified rosin derivatives; terpene resins such as α-pinene and β-pinene; terpene-phenol resins; aliphatic, aromatic, alicyclic and copolymerized petroleum resins, alkylphenyl resins; xylene resins and so on.

A softener is a component which plasticizes/softens a base polymer and maintains an adequate stickiness to the skin. Examples of the softener include polybutene, polyisobutylene, liquid paraffin, higher fatty acid esters such as isopropyl myristate, silicone oil, and vegetable oils such as almond oil, olive oil, camellia oil, persic oil and peanut oil.

In the case of the tape, it is desirable to use, as a support, a material not affecting the release of the pharmacologically active component. A stretchable support and a non-stretchable support are both usable. For example, the support may be selected from films or sheets of synthetic resins such as polyethylene, polypropylene, polybutadiene, ethylene-vinyl acetate copolymer, polyvinyl chloride, polyester, nylon, polyurethane and so on or laminates thereof, porous films and expanded articles thereof, paper, fabrics, non-woven fabrics and so on. The tape can be easily produced in accordance with a publicly known production method. For example, a synthetic rubber-based tape can be produced by heating and mixing a pressure-sensitive adhesive base, a softener and a tackifier at 120 to 160° C. in a mixing machine such as a kneader or a mixer, then adding a pharmacologically active component and isopulegol, 2-(menthoxy)ethanol or 2-methyl-3-(menthoxy)propane-1,2-diol and spreading the resulting mixture directly on a polypropylene or polyester film. Alternatively, the mixture may be once spread on a release-treated paper or film and then covered with a support to thereby press-transfer the mixture onto the support.

An acryl-based tape may be produced by dissolving or dispersing a pressure-sensitive adhesive base, a drug and an absorption promoter optionally together with other compounding components in an appropriate solvent, applying the obtained solution or dispersion directly on a support and then drying to form a lamination layer of 30 to 200 μm in thickness. Alternatively, the solution or dispersion may be applied on a release paper for protective use, dried and then the obtained adhesive layer is brought into close contact with a support. The solvent to be used in the above production method is not particularly restricted, so long as it is compatible with all of the compounding components such as the pressure-sensitive adhesive base and drug. Examples of the solvent include aromatic hydrocarbons such as toluene, benzene and xylene, esters such as ethyl acetate, and halogenated hydrocarbons such as carbon tetrachloride, chloroform and methylene chloride.

Next, formulations of other kinds of external skin formulations such as ointments, gels, creams, gel-type creams, lotions, reservoir type patches, liniments and aerosols will be briefly illustrated. An ointment comprises a pharmacologically active component and isopulegol, 2-(menthoxy)ethanol or 2-methyl-3-(menthoxy)propane-1,2-diol together with at least a higher fatty acid such as myristic acid or an ester thereof, waxes such as whale wax, a surfactant such as polyoxyethylene and hydrocarbons such as hydrophilic vaseline. For example, the ointment is produced by mixing, either at room temperature or elevated temperature, 5 to 15 mass % of the higher fatty acid or an ester thereof, 1 to 10 mass % of the surfactant, 0.5 to 10 mass % of the pharmacologically active component and 0.1 to 20 mass % of isopulegol, 2-(menthoxy)ethanol and 2-methyl-3-(menthoxy)propane-1,2-diol, adding 4 to 10 mass % of the waxes and 50 to 90 mass % of the hydrocarbon, melting the same at elevated temperature or under heating, maintaining the melted mixture at 50 to 100° C., and, after all of the components become transparent and melted, homogeneously mixing the same in a homomixer. Subsequently, the mixture is cooled to room temperature under stirring to give an ointment.

A gel comprises a pharmacologically active component and isopulegol, 2-(menthoxy)ethanol or 2-methyl-3-(menthoxy)propane-1,2-diol together with at least a lower alcohol such as ethanol, water, a gelling agent such as a carboxy vinyl polymer and a neutralizing agent such as triethanolamine. The gel is produced by, for example, adding 0.5 to 5 mass % of the gelling agent to 55 mass % or less of water and allowing the gelling agent to swell. Separately, 0.5 to 10 mass % of the pharmacologically active component and 0.1 to 20 mass % of isopulegol, 2-(menthoxy)ethanol or 2-methyl-3-(menthoxy)propane-1,2-diol are dissolved in a mixture of 40 mass % or less of glycols and 60 mass % or less of a lower alcohol. Both mixtures are combined together and further the neutralizing agent is added thereto to adjust the pH value to 4 to 7. Thus, a gelled preparation is obtained.

A cream comprises a pharmacologically active component and isopulegol, 2-(menthoxy)ethanol or 2-methyl-3-(menthoxy)propane-1,2-diol together with at least a higher fatty acid ester such as a myristate, water, a hydrocarbon such as liquid paraffin and an emulsifier such as a polyoxyethylene alkyl ether. The cream is obtained by mixing the pharmacologically active component, isopulegol, 2-(menthoxy)ethanol or 2-methyl-3-(menthoxy)propane-1,2-diol, the higher fatty acid ester, water, the hydrocarbons and the emulsifier, each in an appropriate amount, and stirring.

A gel-type cream, which has an intermediate nature between gels and creams, is obtained by mixing the individual components of the cream as described above together with a gelling agent such as a carboxy vinyl polymer and a neutralizing agent such as diisopropanolamine, and adjusting the pH value to 4 to 8, preferably 5 to 6.5. The gel-type cream is produced by, for example, dissolving 0.5 to 10 mass % of a pharmacologically active component and 0.1 to 20 mass % of isopulegol, 2-(menthoxy)ethanol or 2-methyl-3-(menthoxy)propane-1,2-diol in a mixture of 25 mass % or less of a higher fatty acid ester and 40 mass % or less of a lower alcohol. Further, 5 mass % or less of the emulsifier is added thereto. Separately, 0.5 to 5 mass % of the gelling agent is added to water and allowed to swell. Next, both mixtures are homogeneously emulsified in a homomixer and then the neutralizing agent is added to give a pH value of 4 to 8.

A lotion comprises a pharmacologically active component and isopulegol, 2-(menthoxy)ethanol or 2-methyl-3-(menthoxy)propane-1,2-diol together with at least a lower alcohol such as ethanol, water and/or glycols. The lotion is produced by mixing the aforesaid pharmacologically active component, isopulegol, 2-(menthoxy)ethanol or 2-methyl-3-(menthoxy)propane-1,2-diol, lower alcohol, water and/or glycol, each in an appropriate amount, and stirring.

A reservoir type patch comprises at least a liner layer (1), a drug-storage layer (2), a drug-release layer (3) and a pressure-sensitive adhesive layer (4). The drug-storage layer (2) comprises a pharmacologically active component and N-mono or disubstituted-p-menthane-3-carboxamide and a base which comprises either (a) at least glycols, a lower alcohol, water and a water-soluble polymer, or (b) at least an aliphatic alcohol and a polyhydric alcohol, or (c) at least paraffins and silicones.

These external skin formulations according to the invention may further contain various pharmacologically acceptable additives such as a stabilizer, an antioxidant, a perfume, a filler, another transdermal absorption promoter or the like, so long as the purpose of the invention is not impaired thereby.

A method for enhancing/controlling the transdermal permeability of a pharmacologically active component, which is selected from among a psychotropic component, an anti-inflammatory component, an analgesic component, an antipyretic component, a whitening component and a hair growth-promoting component, by using, as an active component, the transdermal absorption promoter according to the invention is not specifically restricted, so long as isopulegol, 2-(menthoxy)ethanol or 2-methyl-3-(menthoxy)propane-1,2-diol is compounded. That is, a commonly employed method may be used therefor.

A method for controlling the cooling effect by using, as an active component, the transdermal absorption promoter according to the invention is not specifically restricted, so long as isopulegol, 2-(menthoxy)ethanol or 2-methyl-3-(menthoxy)propane-1,2-diol is compounded. That is, a commonly employed method may be used therefor.

It is known that isopulegol, 2-(menthoxy)ethanol and 2-methyl-3-(menthoxy)propane-1,2-diol, i.e., the active components of the transdermal absorption promoter according to the invention, exert not such a stimulating cooling effect as menthol does but a mild cooling effect. Thus, it is possible to elicit the desired cooling effect by utilizing the effect of these components for promoting the transdermal absorption of a pharmacologically active component.

In addition, by using the desired amount of the above-described warming substance in combination, it is possible to elicit not only the refreshing feeling but also excellent warming effect.

### EXAMPLES

To further illustrate the invention, the following Examples will be given. However, it is to be understood that the invention is not restricted to these Examples.

### Test Example 1

### Evaluation of Effect of Promoting Transdermal Absorption of Paroxetine

(1) Preparation of Samples

As Table 1 shows, hydroxyethylcellulose (HEC) and hydroxypropylcellulose (HPC) were added to purified water and allowed to stand overnight to allow the base to swell. Separately, a principal agent (paroxetine) and various test samples were dissolved in isopropanol (IPA) and homogeneously mixed in the base prepared above. Each mixture was allowed to stand overnight in a dark cool place to give a hydrogel. As a control, purified water was used as a substitute for the test samples.

TABLE 1

| Component | Mass % |
|---|---|
| paroxetine | 1 |
| isopropanol (IPA) | 20 |
| test sample | 2 |
| hydroxyethylcellulose (HEC) | 1 |
| hydroxypropylcellulose (HPC) | 1 |
| purified water | 75 |

Test Samples

[Sample 1] IPG: isopulegol

[Sample 2] 38D: p-menthane-3,8-diol

[Sample 3] CA1: 2-methyl-3-(menthoxy)propane-1,2-diol

[Sample 4] CA5: 2-(menthoxy)ethanol

[Sample 5] CA10: 3-(1-menthoxy)propane-1,2-diol

[Sample 6] d-limonene

[Sample 7] 1-menthol

(2) Skin Permeability Test

A skin specimen extirpated from a hairless mouse was placed in a vertical diffusion cell, with the dermal horny layer side being in the donor side and the dermal basement membrane side being in the receiver side. 16 mL of a phosphate-buffered physiological saline solution (PBS solution) (pH 7.4) was added to the receiver side. The cell was heated in a water bath at 37° C. 30 minutes after starting heating, 1.0 g of a hydrogel, having been heated to 37° C., was applied to the donor side using a needleless syringe. The receiver solution was collected in 1 mL portions at specific time intervals. To the receiver side, the PBS solution was added in 1 mL portions to make up for the collected portions.

The receiver solution thus collected was subjected to high performance liquid chromatography (HPLC) and the amount of permeated paroxetine was calculated.

(3) Measurement Conditions for HPLC

Device: Elite LaChrom System (manufactured by Hitachi Ltd.)

Column: YMS-Pack ODS-A 4.6 mm×150 mm (manufactured by YMC Co., Ltd.)

Eluent: phosphate buffer (pH 3.5)/acetonitrile=35/65 (V/V)

Flow rate: 1.0 mL/min

Measurement wavelength: 235 nm

(4) Results

Table 2 shows the transdermal absorption amount per unit time and unit area (flux) and transdermal absorption speed (lagtime) of each sample solution.

TABLE 2
Fluxes and lagtimes of paroxetine under application
of various monoterpene compounds (n = 5)

| | Flux (μg/cm²/h) | Lagtime (h) |
|---|---|---|
| Control | 5.98 ± 4.36 | 6.25 ± 1.84 |
| 1-Menthol | 185 ± 31.1 | 3.84 ± 1.60 |
| IPG | 214 ± 7.60 | 1.72 ± 0.99 |
| 38D | 15.8 ± 5.90 | 7.79 ± 0.25 |
| CA1 | 167 ± 17.3 | 1.87 ± 0.97 |
| CA10 | 161 ± 10.6 | 5.39 ± 0.99 |
| CA5 | 227 ± 28.9 | 0.85 ± 0.14 |
| d-Limonene | 123 ± 10.3 | 4.29 ± 1.66 |

Table 2 indicates that isopulegol, 2-(menthoxy)ethanol and 2-methyl-3-(menthoxy)propane-1,2-diol show higher transdermal absorption speeds (shorter lagtimes) and larger transdermal absorption amounts per unit time and unit area (fluxes) than other materials.

### Test Example 2

### Evaluation of Effect of Promoting Transdermal Absorption of Antipyrine

(1) Preparation of Samples

As Table 3 shows, hydroxyethylcellulose (HEC) and hydroxypropylcellulose (HPC) were added to purified water and allowed to stand overnight to allow the base to swell. Separately, a principal agent (antipyrine) and various cooling substances were dissolved in isopropanol (IPA) and homogeneously mixed in the base prepared above. Each mixture was allowed to stand overnight in a dark cool place to give a hydrogel.

TABLE 3

| Component | Mass % |
|---|---|
| antipyrine | 2 |
| isopropanol (IPA) | 20 |
| test sample | 2 |
| hydroxyethylcellulose (HEC) | 1 |
| hydroxypropylcellulose (HPC) | 1 |
| purified water | 74 |

Test Samples

[Sample 1] IPG: isopulegol

[Sample 2] 38D: p-menthane-3,8-diol

[Sample 3] CA1: 2-methyl-3-(menthoxy)propane-1,2-diol

[Sample 4] CA5: 2-(menthoxy)ethanol

[Sample 5] CA10: 3-(1-menthoxy)propane-1,2-diol

[Sample 6] d-limonene

[Sample 7] 1-menthol

(2) Skin Permeability Test

The amount of permeated antipyrine was calculated in the same manner as in (2) Skin permeability test and (3) Measurement conditions for HPLC in Test Example 1.

(3) Results

Table 4 shows the transdermal absorption amount per unit time and unit area (flux) and transdermal absorption speed (lagtime) of each sample solution.

TABLE 4
Fluxes and lagtimes of antipyrine under application
of various monoterpene compounds (n = 5)

| | Flux (μg/cm²/h) | Lagtime (h) |
|---|---|---|
| Control | 12.64 ± 2.20 | 3.35 ± 0.04 |
| 1-Menthol | 300.52 ± 34.42 | 0.53 ± 0.02 |
| IPG | 271.94 ± 55.32 | 0.68 ± 0.69 |
| d-Limonene | 67.21 ± 2.61 | 2.00 ± 0.34 |
| CA1 | 150.71 ± 5.46 | 3.20 ± 0.24 |
| CA5 | 224.59 ± 19.56 | 1.36 ± 0.28 |
| CA10 | 100.21 ± 20.34 | 3.00 ± 0.20 |
| 38D | 37.89 ± 8.8 | 4.02 ± 1.30 |

Table 4 indicates that isopulegol, 2-(menthoxy)ethanol and 2-methyl-3-(menthoxy)propane-1,2-diol show higher transdermal absorption speeds (shorter lagtimes) and larger transdermal absorption amounts per unit time and area (fluxes) than the materials except 1-menthol.

### FORMULATION EXAMPLES

Next, formulation examples of pharmaceuticals, cosmetics, etc. comprising the transdermal absorption promoters according to the invention will be illustrated below.

### Example 1

### Lotion

| | |
|---|---|
| Ethanol:purified water (1:1 by mass mixture) | 91.5 mass % |
| Propylene glycol | 5.0 mass % |
| Isopulegol | 3.0 mass % |
| Arbutin | 0.5 mass % |

The above components were mixed together under stirring to give an arbutin-containing lotion.

### Example 2

### Cream

| | |
|---|---|
| Liquid paraffin | 10.0 mass % |
| Medium chain fatty acid triglyceride | 5.0 mass % |
| Polyethylene glycol monostearate | 3.0 mass % |
| Glycerol | 5.0 mass % |
| Carboxy vinyl polymer | 1.0 mass % |
| Diisopropanolamine | 0.4 mass % |
| Methyl paraoxybenzoate | 0.2 mass % |
| Labdenoic acid | 1.0 mass % |
| Isopulegol | 0.5 mass % |
| 2-(Menthoxy)ethanol | 0.5 mass % |
| Purified water | balance |

The above components were mixed together under stirring to give a labdenoic acid-containing cream.

### Example 3

### Ointment

| | | |
|---|---|---|
| White vaseline | 76.0 | mass % |
| Glycerol monostearate | 10.0 | mass % |
| Beef tallow | 10.0 | mass % |
| Silicone oil | 1.0 | mass % |
| Isopulegol | 2.0 | mass % |
| Paroxetine | 1.0 | mass % |

The above components were mixed together under stirring to give a paroxetine-containing ointment.

### Example 4

### Gel

| | | |
|---|---|---|
| Carboxy vinyl polymer | 1.5 | mass % |
| Hydroxypropylcellulose | 2.0 | mass % |
| Ethanol | 17.0 | mass % |
| Purified water | 35.3 | mass % |
| Propylene glycol | 30.0 | mass % |
| Propylene carbonate | 10.0 | mass % |
| Triethanolamine | 0.2 | mass % |
| 2-Methyl-3-(menthoxy)propane-1,2-diol | 3.0 | mass % |
| Indomethacin | 1.0 | mass % |

The above components were mixed together under stirring to give an indomethacin-containing gel.

### Example 5

### Cataplasm

| | | |
|---|---|---|
| Gelatin | 5.0 | mass % |
| Sorbitol | 10.0 | mass % |
| Carboxymethylcellulose | 3.5 | mass % |
| Glycerol | 25.0 | mass % |
| Kaolin | 7.0 | mass % |
| Sodium polyacrylate | 3.0 | mass % |
| Isopulegol | 2.0 | mass % |
| Diclofenac sodium | 1.0 | mass % |
| Purified water | 43.5 | mass % |

The above components were mixed together under heating to give a paste. The paste was spread on a support fabric to give a diclofenac sodium-containing cataplasm.

### Example 6

### Tape

| | | |
|---|---|---|
| Styrene-isoprene-styrene block copolymer | 21.0 | mass % |
| (Kaliflex TR-1107^{R}) | | |
| Polyisobutylene (Vistanex^{R}) | 5.0 | mass % |
| Rosin ester derivative (KE-311^{R}) | 14.0 | mass % |
| Liquid paraffin | 57.0 | mass % |
| Isopulegol | 1.5 | mass % |
| 3-Menthoxypropane-1,2-diol | 0.5 | mass % |
| Felbinac | 1.0 | mass % |

The above components were heated together under stirring. The mixture thus obtained was spread on a support to give a felbinac-containing tape.

### Example 7

### Tape

| | | |
|---|---|---|
| Styrene-isoprene-styrene block copolymer | 20.0 | mass % |
| (Kaliflex TR-1111^{R}) | | |
| Polyisobutylene (Vistanex^{R}) | 12.0 | mass % |
| Liquid paraffin | 30.0 | mass % |
| Rosin ester derivative (Foral 105^{R}) | 30.0 | mass % |
| 2-(Menthoxy)ethanol | 6.0 | mass % |
| p-Menthane-3,8-diol | 1.0 | mass % |
| Loxoprofen sodium | 1.0 | mass % |

The above components were heated together under stirring. The mixture thus obtained was spread on a support to give a loxoprofen-containing tape.

### Example 8

### Reservoir Type Patch

(1) Liner layer: aluminum-laminated polyester film

(2) Drug storage layer: having 4 g of the following gel composition enclosed therein

| | | |
|---|---|---|
| Paroxetine | 3.0 | mass % |
| Isopulegol | 4.0 | mass % |
| 2-Methyl-3-(menthoxy)propane-1,2-diol | 1.0 | mass % |
| Stearyl alcohol | 10.0 | mass % |
| Cetyl alcohol | 10.0 | mass % |
| Behenyl alcohol | 10.0 | mass % |
| Propylene glycol | 22.0 | mass % |
| 1,3-Butylene glycol | 35.0 | mass % |
| Lauryl alcohol | 5.0 | mass % |

(3) Drug release layer: Coatlan

(4) Pressure-sensitive adhesive layer: silicone-based pressure-sensitive adhesive (around the support)

A reservoir type patch, which consisted of the above members (1) to (4), was produced by contacting the release liner to the pressure-sensitive adhesive face to give a laminate.

### Example 9

### Tape

| | | |
|---|---|---|
| Styrene-isoprene-styrene block copolymer | 21.0 | mass % |
| (Kaliflex TR-1107^{R}) | | |
| Polyisobutylene (Vistanex^{R}) | 5.0 | mass % |
| Rosin ester derivative (KE-311^{R}) | 14.0 | mass % |
| Liquid paraffin | 54.5 | mass % |
| Menthol | 1.0 | mass % |
| 2-Methyl-3-(menthoxy)propane-1,2-diol | 1.5 | mass % |
| Felbinac | 3.0 | mass % |

The above components were heated together under stirring. The mixture thus obtained was spread on a support to give a felbinac-containing tape.

### Example 10

### Gel

| | | |
|---|---|---|
| Carboxy vinyl polymer | 1.5 | mass % |
| Hydroxypropylcellulose | 2.0 | mass % |
| Ethanol | 17.0 | mass % |
| Purified water | 35.3 | mass % |
| Propylene glycol | 30.0 | mass % |
| Propylene carbonate | 10.0 | mass % |
| Triethanolamine | 0.2 | mass % |
| 2-Methyl-3-(menthoxy)propane-1,2-diol | 3.0 | mass % |
| Felbinac | 5.0 | mass % |

The above components were mixed together under stirring to give a felbinac-containing gel.

### Example 11

### Gel

| | | |
|---|---|---|
| Carboxy vinyl polymer | 1.5 | mass % |
| Hydroxypropylcellulose | 2.0 | mass % |
| Ethanol | 17.0 | mass % |
| Purified water | 35.3 | mass % |
| Propylene glycol | 30.0 | mass % |
| Propylene carbonate | 10.0 | mass % |
| Triethanolamine | 0.2 | mass % |
| 2-Methyl-3-(menthoxy)propane-1,2-diol | 1.0 | mass % |
| Nonylic acid vanillyl amide | 1.0 | mass % |
| Spilantol | 1.0 | mass % |
| Felbinac | 1.0 | mass % |

The above components were mixed together under stirring to give a felbinac-containing gel.

### Example 12

### Gel

| | | |
|---|---|---|
| Carboxy vinyl polymer | 1.5 | mass % |
| Hydroxypropylcellulose | 2.0 | mass % |
| Ethanol | 17.0 | mass % |
| Purified water | 35.3 | mass % |
| Propylene glycol | 30.0 | mass % |
| Propylene carbonate | 10.0 | mass % |
| Triethanolamine | 0.2 | mass % |
| 2-Methyl-3-(menthoxy)propane-1,2-diol | 1.0 | mass % |
| Isopulegol | 1.0 | mass % |
| Menthol | 1.0 | mass % |
| Felbinac | 1.0 | mass % |

The above components were mixed together under stirring to give a felbinac-containing gel.

### Comparative Example 1

### Gel

| | | |
|---|---|---|
| Carboxy vinyl polymer | 1.5 | mass % |
| Hydroxypropylcellulose | 2.0 | mass % |
| Ethanol | 17.0 | mass % |
| Purified water | 35.3 | mass % |
| Propylene glycol | 30.0 | mass % |
| Propylene carbonate | 10.0 | mass % |
| Triethanolamine | 0.2 | mass % |
| Menthol | 3.0 | mass % |
| Felbinac | 1.0 | mass % |

The above components were mixed together under stirring to give a felbinac-containing gel.

Sensory Evaluation Test

2 g of each of the felbinac-containing gels prepared in Example 12 and Comparative Example 1 were uniformly applied on brachial regions of both arms in healthy panels (10 panels), and their refreshing feeling and drug efficacy were evaluated.

In the initial stage (at five minutes after the application), 6 of 10 panels evaluated that the refreshing feeling and drug efficacy of the gel prepared in Comparative Example 1 were slightly stronger than that of the gel prepared in Example 12. However, 7 of 10 panels at ten minutes after the application and 9 of 10 panels at 30 minutes after the application evaluated that the refreshing feeling and drug efficacy of the gel prepared in Example 12 were more excellent than that of the gel prepared in Comparative Example 1. In addition, 4 of 10 panels evaluated that the irritating feeling was felt in the case of the gel prepared in Comparative Example 1, while no panel evaluated that the irritating feeling was felt in the case of the gel prepared in Example 12.

While the invention has been described in detail and with reference to specific embodiments thereof, it will be apparent to one skilled in the art that various changes and modifications can be made therein without departing from the spirit and scope thereof.

This application is based on Japanese Patent Application No. 2010-137349 filed on Jun. 16, 2010, the entire subject matter of which is incorporated herein by reference.

### INDUSTRIAL APPLICABILITY

By using the transdermal absorption promoter and external skin formulation according to the invention, the transdermal absorption of a drug is remarkably enhanced by isopulegol, 2-(menthoxy)ethanol or 2-methyl-3-(menthoxy)propane-1,2-diol contained therein. Further, the transdermal absorption promoter and external skin formulation according to the invention have little smell and high safety, e.g., not irritating the skin. That is, the external skin formulation comprising the transdermal absorption promoter according to the invention is highly useful in treating various diseases, since it is excellent in safety and usability and enables the quick delivery of a desired drug to a target site or throughout the whole body via the circulatory system.

1. A transdermal absorption promoter, comprising, as an active component, at least one member selected from the group consisting of isopulegol, 2-(menthoxy)ethanol and 2-methyl-3-(menthoxy)propane-1,2-diol.

2. The transdermal absorption promoter according to claim 1, which further comprises at least one component selected from the group consisting of menthol, menthone, camphor, pulegol, cineole, 3-menthoxypropane-1,2-diol, N-alkyl-p-menthane-3-carboxamide, 3-menthoxy-2-methylpropane-1,2-diol, p-menthane-3,8-diol, 3-menthoxypropan-1-ol, 4-1-menthoxybutan-1-ol (menthyl 3-hydroxybutanoate), menthyl 3-hydroxybutanoate, 1-(2-hydroxy-4-methyl-cyclohexyl)ethanone, menthyl lactate, menthol glycerol ketal, N-methyl-2,2-isopropylmethyl-3-methylbutaneamide, menthyl glyoxylate, menthyl succinate, menthyl glutarate, peppermint oil, spearmint oil, eucalyptus oil and mint oil.

3. The transdermal absorption promoter according to claim 1, which further comprises at least one warming substance selected from the group consisting of vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, 4-(1-menthoxy-methyl)-2-(3′-methoxy-4′-hydroxyphenyl)-1,3-dioxolane, red pepper oil, red pepper oleoresin, nonylic acid vanillylamide, jambu oleoresin, Japanese pepper extract, sanshool-I, sanshool-II, sanshoamide, black pepper extract, chavicine, piperine and spilantol.

4. An external skin formulation, which comprises 0.01 to 50 mass % of the transdermal absorption promoter according to claim 1.

5. The external skin formulation according to claim 4, which comprises at least one pharmacologically active component selected from the group consisting of a psychotropic component, an anti-inflammatory component, an analgesic component, an antipyretic component, a whitening component and a hair growth-promoting component.

6. A method for enhancing/controlling the transdermal permeability of at least one pharmacologically active component, which is selected from the group consisting of a psychotropic component, an anti-inflammatory component, an analgesic component, an antipyretic component, a whitening component and a hair growth-promoting component, the method comprising using, as an active component, the transdermal absorption promoter according to claim 1.

7. A method for controlling a cooling effect, which comprises using, as an active component, the transdermal absorption promoter according to claim 1.

8. (canceled)

9. The transdermal absorption promoter according to claim 2, which further comprises at least one warming substance selected from the group consisting of vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, 4-(1-menthoxy-methyl)-2-(3′-methoxy-4′-hydroxyphenyl)-1,3-dioxolane, red pepper oil, red pepper oleoresin, nonylic acid vanillylamide, jambu oleoresin, Japanese pepper extract, sanshool-I, sanshool-II, sanshoamide, black pepper extract, chavicine, piperine and spilantol.

10. An external skin formulation, which comprises 0.01 to 50 mass % of the transdermal absorption promoter according to claim 2.

11. An external skin formulation, which comprises 0.01 to 50 mass % of the transdermal absorption promoter according to claim 3.

12. An external skin formulation, which comprises 0.01 to 50 mass % of the transdermal absorption promoter according to claim 9.

13. The external skin formulation according to claim 10, which comprises at least one pharmacologically active component selected from the group consisting of a psychotropic component, an anti-inflammatory component, an analgesic component, an antipyretic component, a whitening component and a hair growth-promoting component.

14. The external skin formulation according to claim 11, which comprises at least one pharmacologically active component selected from the group consisting of a psychotropic component, an anti-inflammatory component, an analgesic component, an antipyretic component, a whitening component and a hair growth-promoting component.

15. The external skin formulation according to claim 12, which comprises at least one pharmacologically active component selected from the group consisting of a psychotropic component, an anti-inflammatory component, an analgesic component, an antipyretic component, a whitening component and a hair growth-promoting component.

16. A method for enhancing/controlling the transdermal permeability of at least one pharmacologically active component, which is selected from the group consisting of a psychotropic component, an anti-inflammatory component, an analgesic component, an antipyretic component, a whitening component and a hair growth-promoting component, the method comprising using, as an active component, the transdermal absorption promoter according to claim 2.

17. A method for enhancing/controlling the transdermal permeability of at least one pharmacologically active component, which is selected from the group consisting of a psychotropic component, an anti-inflammatory component, an analgesic component, an antipyretic component, a whitening component and a hair growth-promoting component, the method comprising using, as an active component, the transdermal absorption promoter according to claim 3.

18. A method for enhancing/controlling the transdermal permeability of at least one pharmacologically active component, which is selected from the group consisting of a psychotropic component, an anti-inflammatory component, an analgesic component, an antipyretic component, a whitening component and a hair growth-promoting component, the method comprising using, as an active component, the transdermal absorption promoter according to claim 9.

19. A method for controlling a cooling effect, which comprises using, as an active component, the transdermal absorption promoter according to claim 2.

20. A method for controlling a cooling effect, which comprises using, as an active component, the transdermal absorption promoter according to claim 3.

21. A method for controlling a cooling effect, which comprises using, as an active component, the transdermal absorption promoter according to claim 9.
